(19) United States
(12) Patent Application Publication
(10) Pub. No.: US 2007/0060954 A1
(43) Pub. Date: Mar. 15, 2007

(54) Method of using spinal cord stimulation to treat neurological disorders or conditions

(76) Inventors: Tracy Cameron, Toronto (CA); Christopher Chavez, (US)

Correspondence Address: FULBRIGHT & JAWORSKI, L.L.P. (ANS), 2200 ROSS AVENUE, SUITE 2800, DALLAS, TX, 75201-2784, US

(21) Appl. No.: 11/363,383

(22) Filed: Feb. 27, 2006

Related U.S. Application Data
(60) Provisional application No. 60/656,311, filed on Feb. 25, 2005.

(51) Int. Cl. A61N 1/18

(52) U.S. Cl. 607/2

(57) ABSTRACT

The present invention involves methods and systems for using electrical stimulation to treat neurological disorders. More particularly, the method comprises surgically implanting an electrical stimulation lead that is in communication with spinal nervous tissue associated with a first, second, or third cervical vertebral segment to result in spinal nervous tissue stimulation, thus treating a wide variety of neurological disorders.

The present application claims priority to U.S. Provisional Patent Application Ser. No. 60/656,311, filed Feb. 25, 2005, which is incorporated by reference herein in its entirety.

### TECHNICAL FIELD

This invention relates to spinal cord stimulation for treating neurological disorders and related conditions, including at least psychiatric disorders, Alzheimer's, epilepsy, Bell's Palsy, Tourette's Syndrome, Parkinson's Disease, sleep disorders, hypertension, disorders related to blood flow in the brain, depression, anxiety disorders and mood disorders, for example.

### BACKGROUND OF THE INVENTION

Recent estimates indicate that more than 19 million Americans over the age of 18 years experience a depressive illness each year. The American Psychiatric Association recognizes several types of clinical depression, including Mild Depression (Dysthymia), Major Depression, and Bipolar Disorder (Manic-Depression). Major Depression is defined by a constellation of chronic symptoms that include sleep problems, appetite problems, anhedonia or lack of energy, feelings of worthlessness or hopelessness, difficulty concentrating, and suicidal thoughts, for example. Approximately 9.2 million Americans suffer from Major Depression, and approximately 15 percent of all people who suffer from Major Depression take their own lives. Bipolar Disorder involves major depressive episodes alternating with high-energy periods of rash behavior, poor judgment, and grand delusions. An estimated one percent of the American population experiences Bipolar Disorder annually.

Significant advances in the treatment of depression have been made in the past decade. Since the introduction of selective serotonin reuptake inhibitors (SSRIs), i.e., Prozac®, many patients have been effectively treated with anti-depressant medication. New medications to treat depression are introduced almost every year, and research in this area is ongoing. However, an estimated 10 to 30 percent of depressed patients taking an anti-depressant are partially or totally resistant to the treatment. Those who suffer from treatment-resistant depression have almost no alternatives. Thus, there is a need to develop alternative treatments for these patients.

The use of electrical stimulation for treating neurological disease, including such disorders as movement disorders such as Parkinson's disease, essential tremor, dystonia, and chronic pain, has been widely discussed in the literature. It has been recognized that electrical stimulation holds significant advantages over lesioning, because lesioning destroys the nervous system tissue. In many instances, the preferred effect is to modulate neuronal activity. Electrical stimulation permits such modulation of the target neural structures and, equally importantly, does not require the destruction of nervous tissue. Such electrical stimulation procedures include electroconvulsive therapy (ECT), repetitive transcranial (rTMS) magnetic stimulation and vagal nerve stimulation (VNS), for example.

Efforts have been made to treat psychiatric disorders with peripheral/cranial nerve stimulation. Recently, partial benefits with vagus nerve stimulation in patients with depression have been described in U.S. Pat. No. 5,299,569. Another example of electrical stimulation to treat depression is described in U.S. Pat. No. 5,470,846, which discloses the use of transcranial pulsed magnetic fields to treat depression. Yet further, U.S. Pat. No. 5,263,480 describes that stimulation of the vagus nerve may control depression and compulsive eating disorders, and U.S. Pat. No. 5,540,734 teaches stimulation of the trigeminal or glossopharyngeal nerves for psychiatric illness, such as depression.

Various electrical stimulation and/or drug infusion devices have been proposed for treating neurological disorders. Some devices stimulate through the skin, such as electrodes placed on the scalp, for example. Other devices require significant surgical procedures for placement of electrodes, catheters, leads, and/or processing units. These devices may also require an external apparatus that needs to be strapped or otherwise affixed to the skin.

However, despite the aforesaid available treatments, there are patients with neurological disorders that remain treatment refractory to such treatments. For these patients, novel therapies are required. Thus, the present invention provides a novel method of using spinal cord stimulation to treat neurological disorders or conditions.

### BRIEF SUMMARY OF THE INVENTION

The present invention involves methods and systems, for example regarding the therapeutic stimulation concerning a surgically implanted device in communication with spinal nervous tissue associated with one or more of the first, second, or third (C1, C2, or C3) cervical vertebral segments. The device is operated to stimulate (e.g., chemical and/or electrical stimulation) the predetermined spinal nervous tissue, thereby treating one or more neurological disorders. The device can comprise at least one electrode and a pulse generation portion, which, in turn, is operated to stimulate at least one predetermined treatment site.

According to one aspect of the invention, a neurological stimulation system is provided for electrically stimulating a subject's spinal nervous tissue associated with a C1, C2, or C3 cervical vertebral segment to treat one or more neurological disorders. The system includes an electrode or stimulation portion adapted for implantation into a subcutaneous area in communication with the spinal nervous tissue associated with a C1, C2, or C3 cervical vertebral segment. The stimulation portion includes one or more stimulation electrodes adapted to be positioned in the subcutaneous area associated with a C1, C2, or C3 vertebral segment to deliver electrical stimulation pulses to the neuronal tissue. The system also includes a pulse generation source to stimulate the one or more electrodes.

Magnetic stimulation can be provided by internally implanted probes or by externally applied directed magnetic fields, for example. Yet further, thermal stimulation can be provided via implanted probes that are regulated to heat and/or cold temperatures, for example. In other embodiments, ultrasound stimulation is used as a stimulation source, either by itself or in combination with another stimulation source. For example, in certain embodiments of the invention, ultrasound is used to stimulate active tissue by propagating ultrasound in the presence of a magnetic field as described by Norton (2003), herein incorporated by reference in its entirety. Combinations of stimulation sources are used in some embodiments of the invention.

An electrical stimulation system having one or more stimulation electrodes is implanted subcutaneously such that one or more of the stimulation electrodes are in communication with spinal nervous tissue associated with a C1, C2, or C3 cervical vertebral segment. The one or more stimulation electrodes deliver electrical stimulation pulses to the neuronal tissue of one or more of the C1, C2, or C3 cervical vertebral segments, which thereby permanently or temporarily eliminates, reduces, ameliorates or otherwise treats the one or more neurological disorders. This may in turn significantly increase the person's quality of life, in particular aspects of the invention.

In certain embodiments, electrical stimulation of the spinal nervous tissue associated with a C1, C2, or C3 cervical vertebral segment may be provided to effectively treat pain. For example, in certain embodiments, electrical stimulation of the spinal nervous tissue associated with a C1, C2, or C3 cervical vertebral segment may be provided to effectively treat fibromyalgia or other diffuse pain in any one or more regions of the body. As another example, in certain embodiments, electrical stimulation of the spinal nervous tissue associated with a C1, C2, or C3 cervical vertebral segment may be delivered to treat localized, diffuse, or other pain in any one or more regions of the body below the head, such as pain in the neck, shoulders, upper extremities, torso, abdomen, hips, and lower extremities. As another example, in certain embodiments, electrical stimulation of the spinal nervous tissue associated with a C1, C2, or C3 cervical vertebral segment may be delivered to treat Reflex Sympathetic Dystrophy (RSD) pain. As another example, in certain embodiments, electrical stimulation of the spinal nervous tissue associated with a C1, C2, or C3 cervical vertebral segment may decrease the person's overall sensitivity to pain and/or increase the person's overall pain threshold, in certain cases significantly, such that the person experiences “total body” pain relief or other generalized pain relief throughout the body. For example, a person with a relatively low overall pain threshold may experience an elevation of the pain threshold from a relatively hyperalgesic state to a relatively normalized state, with concomitant pain relief throughout the body. Other examples of pain-related applications of electrical stimulation of the spinal nervous tissue associated with a C1, C2, or C3 cervical vertebral segment in certain embodiments include at least the following: (1) treating post-operative pain associated with major surgery, perhaps using a temporary as opposed to a permanent stimulation lead (e.g., to augment or replace opioid analgesia); (2) treating focal pain (e.g., possibly in combination with electrical stimulation of the spinal cord or peripheral structures such as the periostium around the knee or hip); and/or (3) treating pain in elderly patients with severe degenerative spinal or joint conditions (e.g., with additional improvements in sleep, cognition, and mood, for example).

In certain embodiments, possibly in combination with one or more of the benefits described above, electrical stimulation of the spinal nervous tissue associated with a C1, C2, or C3 cervical vertebral segment may be provided to effectively treat impaired motor functioning. For example, in certain embodiments, electrical stimulation of the spinal nervous tissue associated with a C1, C2, or C3 cervical vertebral segment may be provided to effectively treat lack of coordination in the upper or lower extremities (e.g., gait problems). As another example, in certain embodiments, electrical stimulation of the spinal nervous tissue associated with a C1, C2, or C3 cervical vertebral segment may be provided to effectively treat motor disorders such as tremor (e.g., reducing the coarseness of tremor, and Parkinson's disease), dystonia (e.g., reducing the frequency and severity of torticollis or other forms of dystonia), and seizure, for example.

In certain embodiments, possibly in combination with one or more of the benefits described above, electrical stimulation of the area may be provided to effectively treat other neurological disorders for example, but not limited to Developmental Disabilities [e.g., Cerebral Palsy, Mental Retardation, Attention Deficit Disorder (ADD), Pervasive Developmental Disorders and Autistic Spectrum Disorders (e.g., autism and Asperger's disorder), Learning Disabilities (e.g., dyslexia, disorders of motor functions (e.g., dysgraphia, dyspraxia, clumsiness), and nonverbal learning disabilities (e.g., dyscalculia, visuospatial dysfunction, socioemotional disabilities, and ADHD)]; Demyleinating Diseases [e.g., Multiple Sclerosis]; delirium and dementia [e.g., vascular dementia, dementia due to Parkinson's disease, dementia due to HIV disease, dementia due to Huntington's disease, and dementia due to Creutzfeld-Jakob disease; Alzheimer's dementia, multi-infarct dementia, stroke]; affective disorder [e.g., depression, mania, mood disorder, major depressive disorder, bipolar]; movement disorders [e.g, Dyskinesia (e.g., tremor, dystonia, chorea and ballism, tic syndromes (e.g., Tourette's Syndrome), myoclonus, drug-induced movement disorders, Wilson's Disease, Paroxysmal Dyskinesias, Stiff Man Syndrome) and Akinetic-Ridgid Syndromes and Parkinsonism]; ataxic disorders [e.g., disturbances of gait]; substance abuse-related disorders [e.g., alcohol use disorders, amphetamine-use disorders, cannabis-use disorders, caffeine-induced disorders, cocaine-use disorders, inhalant-use disorders, opioid-use disorders, hallucinogen disorders, sedative-use, hypnotic-use, or anxiolytic-use disorders, and polysubstance-use disorders]; sexual dysfunctions [e.g., sexual arousal disorder, male erectile disorder, female dyspareunia, male hypoactive disorder, and female hypoactive disorder]; eating disorders [e.g., overeating disorder, bulimia nervosa, and anorexia nervosa]; anxiety and obsessive compulsive disorder syndromes [e.g., anxiety, panic attacks, post-traumatic stress disorder, agoraphobia, obsessive and compulsive behavior]; impulse control disorders [e.g., pathological gambling, intermittent explosive disorder, kleptomania, and pyromania]; personality disorders (e.g., schizoid personality disorder, paranoid personality disorder, schizotypal personality disorder, borderline personality disorder, narcissistic personality disorder, histrionic personality disorder, obsessive compulsive personality disorder, avoidant personality disorder, dependent personality disorder, and anti-social personality disorder); and other psychiatric disorders [e.g., schizophrenia subtypes, schizoaffective disorder, schizophrenia undifferentiated, delusional disorder, cyclothymic disorder, somatoform disorder, hypochondriasis, dissociative disorder, and depersonalization disorder]; and Chiari I malformation.

In other embodiments of the invention, methods and compositions are useful for the treatment of immune system disorders, such as asthma, for example, and/or cardiac disease, such as vulnerable plaques, for example.

In certain embodiments, electrical stimulation of the spinal nervous tissue associated with a C1, C2, or C3 cervical vertebral segment may effectively treat other conditions including intractable nausea, chronic fatigue, sleep disorders, and/or visceral disorders, such as irritable bowel or areas of the body supplied and controlled mainly by the autonomic nervous system.

In certain embodiments, electrical stimulation of the spinal nervous tissue associated with a C1, C2, or C3 cervical vertebral segment may effectively treat one or more neurological disorders associated with traumatic brain injury (TBI). Physiological conditions associated with TBI that may be treated effectively through electrical stimulation of the spinal nervous tissue associated with a C1, C2, or C3 cervical vertebral segment include, for example, intractable localized, diffuse, or other pain in the head, neck, shoulders, upper extremities, or low back, fibromyalgia or other diffuse pain in one or more regions of the body, or other pain symptoms. Instead of or in addition to such physiological conditions, psychological and other conditions associated with TBI that may be treated effectively through electrical stimulation of the spinal nervous tissue associated with a C1, C2, or C3 cervical vertebral segment include, for example, intractable nausea (e.g., from gastroparesis), sleep disorders, chronic fatigue, behavioral modifications (e.g., lassitude, reduced motivation, depression, emotional distress, irritability, aggression, anxiety, erratic mood swings, personality changes, and loss of enjoyment), sexual dysfunction, and other conditions. Instead of or in addition to physiological, psychological, and other conditions such as those described above, conditions associated with TBI that may be treated effectively through electrical stimulation of the spinal nervous tissue associated with a C1, C2, or C3 cervical vertebral segment include decreased cognitive functioning in the form of, for example, impaired memory (e.g., short-term memory, visual memory, and auditory memory), reduced attention and concentration, and reduced information processing capacity (e.g., learning capacity, ability to process complex information, ability to operate simultaneously on different information, ability to rapidly shift attention, ability to plan and sequence, visuomotor capability, auditory language comprehension, and verbal fluency), for example.

An embodiment of the invention is a method of treating hypertension in a patient comprising the steps of surgically implanting in the patient a stimulation system in communication with spinal nervous tissue at one or more areas associated with the first, second, or third cervical vertebral segment; operating the system to stimulate the spinal nervous tissue; and treating hypertension in the patient.

Another embodiment of the invention is a method of treating a migraine headache in a patient comprising the steps of surgically implanting in the patient a stimulation system in communication with spinal nervous tissue at one or more areas associated with the first, second, or third cervical vertebral segment; operating the system to stimulate the spinal nervous tissue; and treating the migraine headache in the patient.

In one embodiment of the invention, the neurological disease or condition is assessed before, during, and/or after stimulating the spinal nervous tissue associated with the first, second, or third cervical vertebral segment. As used herein, the assessing may be monitoring, testing, imaging, assaying, or evaluating according to methods known to one with skill in the art. In one embodiment, a patient's own self-assessment is used to determine the effectiveness of the treatment. For example, a migraine headache is treated by stimulating the spinal nervous tissue associated with the first, second, or third cervical vertebral segment. After treatment, the patient is interviewed to determine the extent of pain relief. In another embodiment, a patient is treated for hypertension by stimulating the spinal nervous tissue associated with the first, second, or third cervical vertebral segment, and the patient's blood pressure is monitored. In certain embodiments, the patient is monitored by a sphygmomanometer. In certain embodiments, the patient is monitored with an ambulatory blood pressure monitor. In certain embodiments, secondary effects of hypertension are assessed by echocardiography, chest X-ray, or electron beam computed tomography [CT] scan, for example. In other embodiments of the invention, cerebral blood flow is assessed by MRI, PET, or Laser Doppler Flowmetry, for example.

In another embodiment of the invention, the neurological disorder or condition is assessed by motor examination, cranial nerve examination, and/or neuropsychological tests (i.e., Minnesota Multiphasic Personality Inventory, Beck Depression Inventory, or Hamilton Rating Scale for Depression, for example). In addition to the above examinations, imaging techniques can be used to determine normal and abnormal brain function that can result in disorders. Functional brain imaging allows for localization of specific normal and abnormal functioning of the nervous system. This includes exemplary electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET), which can be utilized to localize brain function and dysfunction.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A and 1B illustrate example electrical stimulation systems.

FIGS. 2A-2I illustrate example electrical stimulation leads that may be used in the present invention.

FIGS. 3A and 3B illustrate a spinal cord diagram.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

As used herein the term “affective disorders” refers to a group of disorders that are commonly associated with co-morbidity of depression and anxiety symptoms.

As used herein the term “anxiety” refers to an uncomfortable and unjustified sense of apprehension that may be diffuse and unfocused and is often accompanied by physiological symptoms.

As used herein the term “anxiety disorder” refers to or connotes significant distress and dysfunction due to feelings of apprehension, guilt, fear, etc. Anxiety disorders include, but are not limited to panic disorders, posttraumatic stress disorder, obsessive-compulsive disorder and phobic disorders, for example.

As used herein the term “subcutaneous” refers to an area underneath the skin that is appropriate for implantation of an electrode or stimulation portion adapted for implantation. In one aspect, the lead is implanted subcutaneously and in communication with the spinal nervous tissue associated with a C1, C2, or C3 cervical vertebral segment. In another embodiment, the pulse generation portion is implanted subcutaneously. In one embodiment, the pulse generation portion is transcutaneously in communication with the stimulation portion or electrode. In “transcutaneous”, electrical nerve stimulation (TENS) the stimulation source is external to the patient's body, and may be worn in an appropriate fanny pack or belt, and the electrode or stimulation portion is in communication with the pulse generation portion, either remotely or directly. In another embodiment, the stimulation is percutaneous. In “percutaneous” electrical nerve stimulation (PENS), needles are inserted to an appropriate depth around or immediately adjacent to a predetermined stimulation site, and then stimulated.

As used herein, the use of the words “epidural space” or “spinal epidural space” is known to one with skill in the art, and refers to an area in the interval between the dural sheath and the wall of the spinal canal. It is contemplated that electrode or stimulation portion may be implanted in the epidural space, for example. As used herein, the term “subdural” refers to the space between the dura mater and arachnoid membrane. In certain embodiments of the invention, a stimulation portion or electrode may be implanted in the subdural space.

As used herein, the term “in communication” refers to the at least one electrode or stimulation portion being adjacent, in the general vicinity, in close proximity, or directly next to and/or directly on the predetermined stimulation site, such as a level or area of the spinal cord associated with cervical vertebral segments. Thus, one of skill in the art understands that the lead is “in communication” with the nervous tissue or spinal cord associated with a cervical vertebral segment if the stimulation results in a modulation of neuronal activity resulting in the desired response, such as modulation of the neurological disorder, for example.

The terms “mammal,” “mammalian organism,” “subject,” or “patient” are used interchangeably herein and include, but are not limited to, humans, dogs, cats, horses and cows, for example. The preferred patients are humans.

As used herein the term “modulate” refers to the ability to regulate neuronal activity positively or negatively neuronal activity, including but not limited to, neuronal activity via stimulation of the spinal cord or spinal nervous tissue associated with the cervical vertebral segments that innervates at least the ointracranial vessels, lacrimal glands, ciliary ganglion, parotid glands, the larynx, trachea, bronchi, lungs, pulmonary plexus, cardiac plexus, and the heart. Further, the term “modulate” can be used to refer to an increase, decrease, masking, altering, overriding or restoring neuronal activity, including but not limited to, neuronal activity associated with the cervical nerve roots. Modulation of neuronal activity, such as that associated with the cervical nerve roots, for example, can affect pain and/or neurological activity, among other effects.

As used herein the term “mania” or “manic” refers to a disordered mental state of extreme excitement.

As used herein the term “mood” refers to an internal emotional state of a person.

As used herein the term “mood disorder” is typically characterized by pervasive, prolonged, and disabling exaggerations of mood and affect that are associated with behavioral, physiologic, cognitive, neurochemical and psychomotor dysfunctions. The major mood disorders include, but are not limited to major depressive disorder (also known as unipolar disorder), bipolar disorder (also known as manic depressive illness or bipolar depression), dysthymic disorder. Other mood disorders may include, but are not limited to, major depressive disorder, psychotic; major depressive disorder, melancholic; major depressive disorder, seasonal pattern; postpartum depression; brief recurrent depression; late luteal phase dysphoric disorder (premenstrual dysphoria); and cyclothymic disorder, for example.

As used herein, the term “neurology” or “neurological” refers to conditions, disorders, and/or diseases that are associated with the nervous system. The nervous system comprises two components, the central nervous system, which is comprised of the brain and the spinal cord, and the peripheral nervous system, which is comprised of ganglia and the peripheral nerves that lie outside the brain and the spinal cord. One of skill in the art realizes that the nervous system may be separated anatomically, but functionally they are interconnected and interactive. Yet further, the peripheral nervous system is divided into the autonomic system (parasympathetic and sympathetic), the somatic system, and the enteric system. Thus, any condition, disorder and/or disease that affects any component or aspect of the nervous system (either central or peripheral) is referred to as a neurological condition, disorder and/or disease. As used herein, the term “neurological” or “neurology” encompasses the terms “neuropsychiatric” or “neuropsychiatry” and “neuropsychological” or “neuropsychological”. Thus, a neurological disease, condition, or disorder includes, but is not limited to, cognitive disorders, affective disorders, movement disorders, mental disorders, pain disorders, sleep disorders, etc. For example, neurological disorders include hypertension, migraine headaches, depression, and epilepsy.

As used herein, the term “neuronal” refers to a neuron that is a morphologic and functional unit of the brain, spinal column, and peripheral nerves.

As used herein, the term “pharmaceutical” refers to a chemical or agent that is used as a drug. Thus, the term pharmaceutical and drug are interchangeable, in specific embodiments of the invention.

As used herein, the term “stimulate” or “stimulation” refers to electrical and/or chemical modulation of selected cervical nervous tissue, cervical nerve roots, cervical segments, cervical levels, or areas of the spinal cord associated with a cervical vertebral segment.

The phrase “spinal cord stimulation” as used herein includes stimulation of any spinal nervous tissue, including spinal neurons, accessory neuronal cells, nerves, nerve roots, nerve fibers, or tissues, that are associated with the spinal cord. It is contemplated that spinal cord stimulation may comprise stimulation of one or more areas associated with a cervical vertebral segment.

As used herein, “spinal nervous tissue” refers to nerves, neurons, neuroglial cells, glial cells, neuronal accessory cells, nerve roots, nerve fibers, nerve rootlets, parts of nerves, nerve bundles, mixed nerves, sensory fibers, motor fibers, dorsal root, ventral root, dorsal root ganglion, spinal ganglion, ventral motor root, general somatic afferent fibers, general visceral afferent fibers, general somatic efferent fibers, general visceral efferent fibers, grey matter, white matter, the dorsal column, the lateral column, and/or the ventral column associated with the spinal cord. Spinal nervous tissue includes “spinal nerve roots,” which comprise the 31 pairs of nerves that emerge from the spinal cord. Spinal nerve roots may be cervical nerve roots, cervical nerve roots, and lumbar nerve roots, for example.

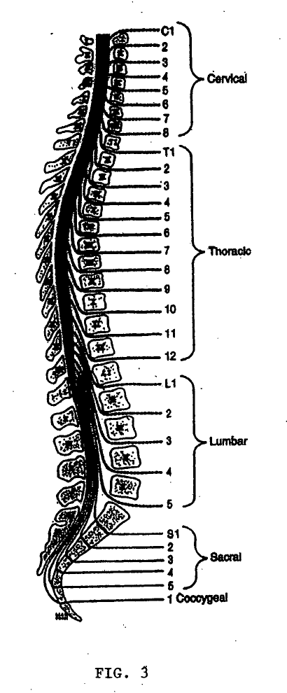

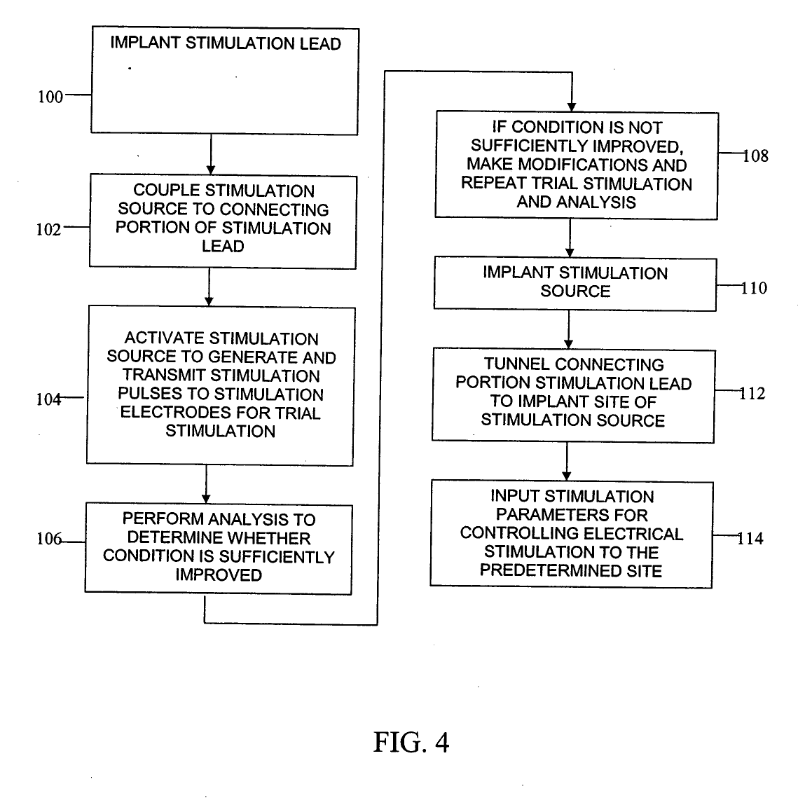

As used herein, “spinal nervous tissue associated with a cervical vertebral segment,” or “nervous tissue associated with a cervical vertebral segment” or “spinal cord associated with a cervical segment or level” includes any spinal nervous tissue associated with a cervical vertebral level or segment, which can include at least one cervical nerve root and tissue associated therewith, for example. Those of skill in the art are aware that the spinal cord and tissue associated therewith are associated with cervical, thoracic, and lumbar vertebrae. In the present invention, the spinal cord or spinal tissue that is stimulated is associated with at least one or more of the cervical vertebra. See also FIGS. 3A and 3B. As used herein, C1 refers to cervical vertebral segment 1 or the first vertebral segment, C2 refers to cervical vertebral segment 2 or the second vertebral segment, C3 refers to cervical vertebral segment 3 or the third vertebral segment, C4 refers to cervical vertebral segment 4 or the fourth vertebral segment, C5 refers to cervical vertebral segment 5 or the fifth vertebral segment, C6 refers to cervical vertebral segment 6 or the sixth vertebral segment, and C7 refers to cervical vertebral segment 7 or the seventh vertebral segment, unless otherwise specifically noted.

As used herein, “atlas” may refer to the first cervical vertebra. The atlas is a ring of bone made up of two lateral masses joined at the front and back by the anterior arch and the posterior arch. As used herein, “axis” may refer to the second cervical vertebra. The axis is a blunt tooth-like process that projects upward. The “axis” is also referred to as the ‘dens’ (Latin for ‘tooth’) or odontoid process. The dens provides a type of pivot and collar allowing the head and atlas to rotate around the dens.

As used herein, “cervical nerve roots,” “nerves or nerve roots associated with a cervical vertebral segment,” or “nerve roots associated with a cervical vertebral level,” refer to nerves associated with levels, or segments of the cervical vertebrae. There are eight total cervical nerve roots, and seven cervical vertebrae. Cervical nerve roots are numbered according to the vertebrae above which they emerge. Thus, one with skill in the art realizes that the C1 nerve root emerges above the C1 vertebra, the C2 nerve root emerges between the C1 vertebra and C2 vertebra, the C3 nerve root emerges between the C2 vertebra and C3 vertebra, and so on. The C8 nerve root emerges below the C7 vertebra and above the T1 vertebra. One with skill in the art also would be aware that the C1 nerve root comes out between occipital and atlas, the C2 nerve root comes out between atlas and axis, and the C3 nerve root comes out between axis and C3 vertebra. One with skill in the art realizes that due to aberrants (missing ribs) or genetic variations, the exiting of the nerve may be altered in individual subjects, and the above serves as a general guideline. The C1 nerve is also known as the suboccipital nerve, and exits the spinal cord between the skull and the first cervical vertebra, the atlas. It supplies muscles around the suboccipital triangle including the rectus capitis posterior major, obliquus capitis superior, and obliquus capitis inferior.

As used herein, the term “treating” and “treatment” refers to stimulating certain nervous tissue of the spinal cord so that the subject has at least an improvement in the disease, for example, beneficial or desired clinical results. For purposes of this invention, beneficial or desired clinical results include, but are not limited to, alleviation of symptoms, alleviation of pain, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

II. Electrical Stimulation Systems

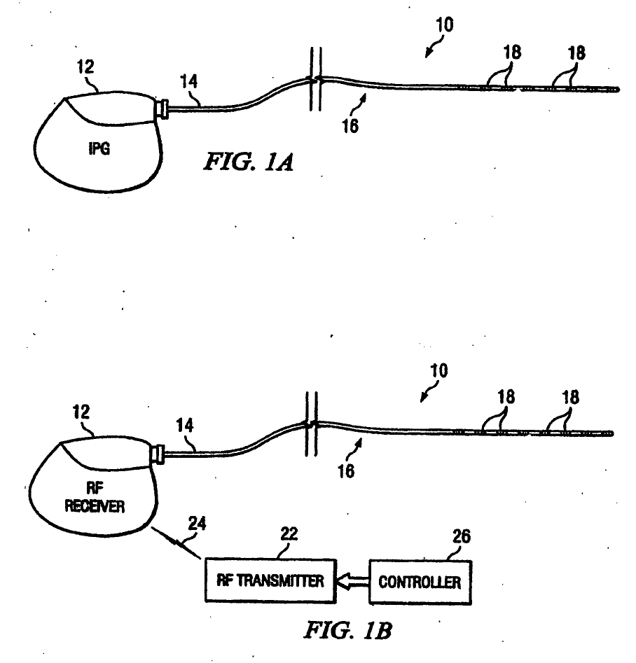

FIGS. 1A and 1B illustrate example electrical stimulation systems 10 used to stimulation to a target a predetermined site. Stimulation system 10 generates and applies a stimulus to a target area that is in communication with a predetermined site in which stimulation of such site will reduce or alleviate a neurological condition and/or disorder.

In general terms, stimulation system 10 includes an implantable pulse generation portion (e.g., electrical stimulation source) 12 and an implantable stimulation portion (e.g., electrical stimulation lead, or electrode) 14 for applying the stimulation signal to the target the spinal cord. In operation, both of these primary components are implanted in the person's body. Pulse generation portion 12 is coupled to a connecting portion 16 of electrical stimulation portion 14. In certain other embodiments, pulse generation source 12 is not coupled directly to stimulation portion 14 and pulse generation source 12 instead communicates with stimulation portion 14 via a wireless link. For example, such a stimulation system 10 is described in the following patents U.S. Pat. Nos. 6,748,276; 5,938,690, each of which is incorporated by reference in its entirety. In certain other embodiments, pulse generation source 12 and electrodes 18 are contained in an “all-in-one” microstimulator or other unit, such as a Bion® microstimulator manufactured by Advanced Bionics Corporation. A doctor, the patient, or another user of pulse generation source 12 may directly or indirectly input signal parameters for controlling the nature of the electrical stimulation provided. Whether pulse generation source 12 is coupled directly to or embedded within the stimulation portion 14, pulse generation source 12 controls the stimulation pulses transmitted to one or more stimulation electrodes 18 located on a stimulating portion 14, positioned in communication with a predetermined site to stimulate spinal nerves, according to suitable stimulation parameters (e.g., duration, amplitude or intensity, frequency, pulse width, etc.).

In one embodiment, as shown in FIG. 1A, pulse generation source 12 includes an implantable pulse generator (IPG). One of skill in the art is aware that any commercially available implantable pulse generator can be used in the present invention, as well as a modified version of any commercially available pulse generator. Thus, one of skill in the art would be able to modify an IPG to achieve the desired results. An exemplary IPG is one that is manufactured by Advanced Neuromodulation Systems, Inc., such as the Genesis® System, part numbers 3604, 3608, 3609, and 3644. Another example of an IPG is shown in FIG. 1B, which shows stimulation source 12 including an implantable wireless receiver. An example of a wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIG. 1B by wireless link symbol 24. A doctor, the patient, or another user of pulse generation source 12 may use a controller 26 located external to the person's body to provide control signals for operation of pulse generation source 12. Controller 26 provides the control signals to wireless transmitter 22, wireless transmitter 22 transmits the control signals and power to the wireless receiver of pulse generation source 12, and pulse generation source 12 uses the control signals to vary the signal parameters of electrical signals transmitted through stimulation portion 14 to the stimulation site. An example wireless transmitter 122 may be one manufactured by Advanced Neuromodulation Systems, Inc., such as the Renew® System, part numbers 3508 and 3516.

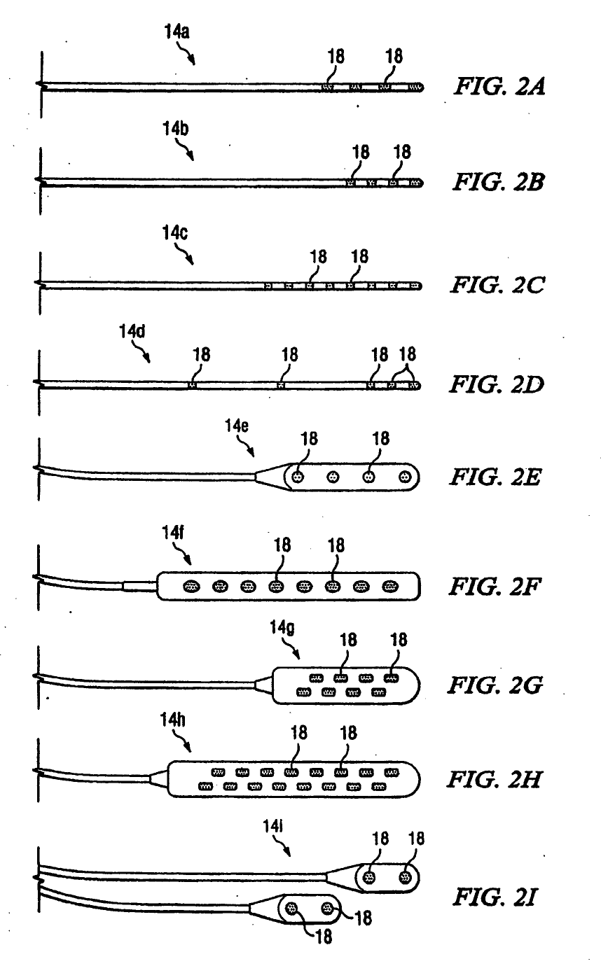

FIGS. 2A-2I illustrate example electrical stimulation leads 14 that may be used to provide electrical stimulation to an area of the spinal cord. As described above, each of the one or more leads 14 incorporated in stimulation system 10 includes one or more electrodes 18 adapted to be positioned near the target cervical segment and used to deliver electrical stimulation energy to the target cervical segment in response to electrical signals received from pulse generation source 12. A percutaneous lead 14, such as example leads shown in FIGS. 2A-2D, includes one or more circumferential electrodes 18 spaced apart from one another along the length of lead 14. An example of an eight-electrode percutaneous lead is an OCTRODE® lead manufactured by Advanced Neuromodulation Systems, Inc. A stimulation system such as is described in U.S. Pat. No. 6,748,276 is also contemplated. Circumferential electrodes 18 emit electrical stimulation energy generally radially in all directions.

A laminotomy, paddle, or surgical stimulation lead 14, such as example stimulation leads 14E-I, includes one or more directional stimulation electrodes 18 spaced apart from one another along one surface of stimulation lead 14. An example of an eight-electrode, two column laminotomy lead is a LAMITRODE® and C-series LAMITRODE® 44 leads manufactured by Advanced Neuromodulation Systems, Inc. Directional stimulation electrodes 18 emit electrical stimulation energy in a direction generally perpendicular to the surface of stimulation lead 14 on which they are located.

Although various types of stimulation leads 14 are shown as examples, the present invention contemplates stimulation system 10 including any suitable type of stimulation portion 14 in any suitable number. In addition, stimulation portion 14 may be used alone or in combination. For example, medial or unilateral stimulation of the predetermined site may be accomplished using a single stimulation portion 14 implanted in communication with the predetermined site in one side of the head, while bilateral electrical stimulation of the predetermined site may be accomplished using two stimulation portion 14 implanted in communication with the predetermined site in opposite sides of the head. Yet further, in certain embodiments for stimulation of cervical spinal tissue, the stimulation portion can be parallel to the spinal cord or the stimulation portion can be perpendicular to the spinal cord.

Whether using percutaneous leads, laminotomy leads, or some combination of both, the leads are coupled to one or more conventional neurostimulation devices, or pulse generation portion. The devices can be totally implanted systems and/or radio frequency (RF) systems. An example of an RF system is a Renew® system manufactured by Advanced Neuromodulation Systems, Inc.

A contemplated stimulation system may have no leads, with the electrodes directly connected to the pulse generator. Alternatively, in another embodiment, a stimulation system with flexible leads is also contemplated. One with skill in the art realizes that the methods of the present invention are appropriate for use with any stimulation device capable of providing stimulation to spinal nervous tissue. In other embodiments, a transcutaneous electrical nerve stimulator (TENS) is envisioned for use in the method and systems of the invention.

In certain embodiments, the stimulation may be continuous or administered as needed. In other embodiment, the stimulation is randomly generated in order to modulate effects such as brain or nerve plasticity.

The preferred neurostimulation systems should allow each electrode to be defined as a positive, a negative, or a neutral polarity. For each electrode combination (i.e., the defined polarity of at least two electrodes having at least one cathode and at least one anode), an electrical signal can have at least a definable amplitude (i.e., voltage), pulse width, and frequency, where these variables may be independently adjusted to finely select the sensory transmitting nerve tissue required to inhibit transmission of neuronal signals. Generally, amplitudes, pulse widths, and frequencies are determinable by the capabilities of the neurostimulation systems.

Voltage or intensity that can be used may include a range from about 1 millivolt to about 1 volt or more, e.g., 0.1 volt to about 50 volts, e.g., from about 0.2 volt to about 20 volts and the frequency may range from about 1 Hz to about 25000 Hz, about 50 Hz-3,000 Hz, about 1 Hz to about 1000 Hz, e.g., from about 2 Hz to about 100 Hz in certain embodiments. The pulse width may range from about 1 microsecond to about 2000 microseconds or more, e.g., from about 10 microseconds to about 2000 microseconds, e.g., from about 15 microseconds to about 1000 microseconds, e.g., from about 25 microseconds to about 1000 microseconds. The electrical output may be applied for at least about 1 millisecond or more, e.g., about 1 second, e.g., about several seconds, where in certain embodiments the stimulation may be applied for as long as about 1 minute or more, e.g., about several minutes or more, e.g., about 30 minutes or more may be used in certain embodiments.

It is envisaged that the patient will require intermittent assessment with regard to patterns of stimulation. Different electrodes on the lead can be selected by suitable computer programming, such as that described in U.S. Pat. No. 5,938,690, which is incorporated by reference here in full. Utilizing such a program allows an optimal stimulation pattern to be obtained at minimal voltages. This ensures a longer battery life for the implanted systems.

III.Implantation of Electrical Stimulation Systems

One technique that offers the ability to affect neuronal function is the delivery of electrical stimulation for neuromodulation directly to target tissues via an implanted system having an electrode. Another technique that offers the ability to affect neuronal function is the delivery of electrical stimulation for neuromodulation directly to target tissues via an implanted system having a stimulation lead. The electrode assembly of the stimulation system may be one electrode, multiple electrodes, or an array of electrodes in or around the target area. The proximal end of the probe or lead is coupled to system to stimulate the target site. Thus, the probe or lead is coupled to an electrical signal source which, in turn, is operated to stimulate the predetermined treatment site.

In certain embodiments, the predetermined site or treatment site is spinal nervous tissue associated with a C1, C2, or C3 cervical vertebral segment. Yet further, one of skill in the art realizes that stimulation of spinal tissue associated with C1, C2, or C3 cervical vertebral segment can result in stimulation of cranial nerves, e.g., olfactory nerve, optic, nerve, oculomoter nerve, trochlear nerve, trigeminal nerve, abducent nerve, facial nerve, vestibulocochlear nerve, glossopharyngeal nerve, vagal nerve, accessory nerve, and the hypoglossal nerve.

Techniques for implanting electrodes are well known in the art. For example, stimulation electrodes 18 may be positioned in various body tissues and in contact with various tissue layers; for example, subdural, subarachnoid, epidural, cutaneous, transcutaneous and subcutaneous implantation is employed in some embodiments. The electrodes are carried by two primary vehicles: a percutaneous leads and a laminotomy lead. These electrodes may be placed parallel to the spinal cord, for example placed on the dorsal side, or perpendicular to the spinal cord.

For spinal cord stimulation, percutaneous leads commonly have two or more equally-spaced electrodes, which are placed above the dura layer through the use of a Touhy-like needle. For insertion, the Touhy-like needle is passed through the skin, between desired vertebrae, to open above the dura layer. For unilateral stimulation, percutaneous leads are positioned on a side of a spinal column corresponding to the “afflicted” side of the body, as discussed above, and for bilateral stimulation, a single percutaneous lead is positioned along the patient midline (or two or more leads are positioned on each side of the midline). An example of an eight-electrode percutaneous lead is an OCTRODE® lead manufactured by Advanced Neuromodulation Systems, Inc. A stimulation system such as is described in U.S. Pat. No. 6,748,276 is also contemplated.

Laminotomy leads have a paddle configuration and typically possess a plurality of electrodes (for example, two, four, eight, or sixteen) arranged in one or more columns. An example of a sixteen-electrode laminotomy lead is shown in FIG. 2.

Implanted laminotomy leads are commonly transversely centered over the physiological midline of a patient. In such position, multiple columns of electrodes are well suited to address both unilateral and bilateral pain, where electrical energy may be administered using either column independently (on either side of the midline) or administered using both columns to create an electric field which traverses the midline. A multi-column laminotomy lead enables reliable positioning of a plurality of electrodes, and in particular, a plurality of electrode columns that do not readily deviate from an initial implantation position.

Laminotomy leads require a surgical procedure for implantation. see for example, US Application No. US20050033393, which is incorporated by reference in its entirety. The surgical procedure, or partial laminectomy, requires the resection and removal of certain vertebral tissue to allow both access to the dura and proper positioning of a laminotomy lead. The laminotomy lead offers a more stable platform, which is further capable of being sutured in place, that tends to migrate less in the operating environment of the human body. Unlike the needle-delivered percutaneous leads, laminotomy leads have a paddle configuration. The paddle typically possess a plurality of electrodes (for example, two, four, eight, or sixteen) arranged in some pattern, for example, columns. An example of an eight-electrode, two column laminotomy lead is a LAMITRODE® and C-series LAMITRODE® 44 leads manufactured by Advanced Neuromodulation Systems, Inc. In the context of conventional spinal cord stimulation, the surgical procedure, or partial laminectomy, requires the resection and removal of certain vertebral tissue to allow both access to the dura and proper positioning of a laminotomy lead. Depending on the position of insertion, however, access to the dura may only require a partial removal of the ligamentum flavum at the insertion site. In a preferred embodiment, two or more laminotomy leads are positioned within the epidural space of C1, C2, or C3, or both. The leads may assume any relative position to one another.

In addition to the use of these leads, the present invention can also utilize a Bion® stimulation system manufactured by Advanced Bionics Corporation. Thus, the present invention can utilize any type of lead and/or stimulation system to stimulate a predetermined cervical vertebral segment neuronal tissue site.

The implant site of the pulse generation source may be a subcutaneous pocket formed to receive and house pulse generation source 12. The implant site is usually positioned a distance away from the insertion site, such as in the chest, buttocks, or another suitable location. However, a suitably small pulse generation source 12 may be used to allow pulse generation 12 to be implanted at or very near the stimulation site. Connecting portion 16 of electrical stimulation portion 14 extends from the electrical lead insertion site to the implant site at which pulse generation source 12 is implanted. Where appropriate, an extension may be used to connect electrical stimulation portion 14 to pulse generation source 12. A doctor, the patient, or another user of pulse generation source 12 may thereafter directly or indirectly input or modify one or more stimulation parameters to specify the nature of the stimulation provided

In certain embodiments, stimulation portion 14 is implanted in or under the person's skin (i.e., in the epidermis, dermis, or subcutaneous tissue) surrounding, overlying, or otherwise proximate the predetermined site, as described for example in U.S. Application No. 60/547,506, filed Feb. 25, 2004, entitled “SYSTEM AND METHOD FOR NEUROLOGICAL STIMULATION OF PERIPHERAL NERVES TO TREAT LOW BACK PAIN” is hereby incorporated by reference in its entirety.

In other embodiments, stimulation portion 14 is implanted in tissue surrounding, overlying, or otherwise proximate the predetermined cervical vertebral segment site. For example, stimulation portion 14 may be implanted in the epidermis, the dermis, or the subcutaneous tissue proximate the predetermined cervical segment site. In a particular embodiment, stimulation portion 14 is implanted approximately one centimeter deep, in a tissue plane lying between the dermal and subdermal tissues. In general, the closer electrodes 18 are to the surface of the skin, the less likely the stimulation will cause contractions of the underlying muscles.

Preferably, electrical stimulation portion 14 should be anchored using a suitable anchoring technique. Anchoring electrical stimulation portion 14 for spinal nerve stimulation may be a challenge due to the slight differences between the anatomies of patients, in particular the tissue planes in which electrical stimulation portion 14 is to be implanted. In contrast, anchoring an electrical stimulation portion 14 used for spinal cord stimulation as it exits the epidural space may be more straightforward. In a particular embodiment, two anchors are utilized to anchor electrical stimulation portion 14—a “butterfly” anchor such as one manufactured by Advanced Neuromodulation Systems, Inc., part number 64-1105, and a “long” anchor such as one manufactured by Advanced Neuromodulation Systems, Inc., part number 64-1106. After placement of the stimulation portion is finalized, a small incision is made at the point where needle 104 exits the skin and dissection is performed down to the fascial plane. The wings or tabs of the butterfly anchor are cut off and the butterfly anchor is placed on the lead body and sutured to the dermal or subdermal tissue layer superficially and perpendicular to the surface of the skin. The long anchor is then threaded onto electrical stimulation portion 14. Electrical stimulation portion 14 is looped around to the fascial surface with the long anchor positioned flat against the fascial plan and then sutured to the fascia. Once the anchors are secured, preferably after complete implantation of electrical stimulation portion 14 and pulse generation source 12, the anchoring pocket can be closed. Although a particular anchoring technique is described in detail, other embodiments may involve other suitable anchoring techniques according to particular needs.

FIG. 4 illustrates an example method of implanting stimulation system 10, described above, into a person's body with stimulation portion located in communication with a predetermined cervical segment site to treat a neurological disorder or condition. At step 100, one or more stimulation portion so that the stimulation portion is in communication with the predetermined cervical segment site (for the purposes described herein and as those skilled in the art will recognize, when an embedded stimulation system, such as the Bion®, is used, it is positioned similar to positioning the stimulation portion). Techniques for implanting stimulation portions are known to those skilled in the art. In certain embodiments, as described above, one or more stimulation portions or electrodes are positioned in communication with the cervical segment tissue site. At step 102, if necessary, pulse generation source may be coupled directly to a connecting portion of a stimulation portion. Alternatively, as described above and if necessary, pulse generation source may not be coupled directly to a stimulation portion and may instead be coupled to a stimulation portion via an appropriate wireless link. Of course, as those skilled in the art know, an embedded stimulation system will not need to be so coupled.

Intra-implantation trial stimulation may be conducted at steps 104 through 108. Alternatively, the method may proceed from step 102 to 110. At step 104, pulse generation source is activated to generate and transmit stimulation pulses via one or more electrodes. At step 106, informal subjective questioning of the person, formal subjective testing and analysis according to one or more neuropsychological test batteries, or other analysis may be performed to determine whether the one or more neurological disorder, or other conditions are sufficiently improved through the intra-implantation trial stimulation. If the one or more neurological, or other conditions are not sufficiently improved, one or more stimulation parameters may be adjusted, a stimulation portion may be moved incrementally or even re-implanted, or both of these modifications may be made at step 108 and the trial stimulation and analysis repeated until the one or more neurological conditions are sufficiently improved. Once the stimulation parameters have been properly set and stimulation portion has been properly positioned such that the one or more physiological, psychological, or other conditions are sufficiently improved, intra-implantation trial stimulation is complete. One of skill in the art is aware that other types of intra-implantation trailing methods or stimulation trails can be used in the present invention, for example, but not limited to transcutaneous electrical nerve stimulation (TENS), transmagnetic stimulation (TMS), nerve blocks, etc.

Once a stimulation portion has been properly implanted and secured, and any trial stimulation completed, if necessary, a pulse generation source is implanted at step 110. Techniques for implanting stimulation sources such as a pulse generation source are known to those skilled in the art. For non-embedded systems, the implant site is typically a subcutaneous pocket formed to receive and house a pulse generation source. The implant site is usually located some distance away from the insertion site, such as in or near the upper chest or buttocks. Where stimulation portion includes a connecting portion, the connecting portion may be tunneled, at least in part, subcutaneously to the implant site of a pulse generating source at step 112. At step 114, a doctor, the patient, or another user of the pulse generation source may directly or indirectly input stimulation parameters for controlling the nature of the electrical stimulation provided to the predetermined cervical segment tissue site, if not already set during any intra-implantation trial stimulation period. Where appropriate, post-implantation trial stimulation may be conducted over about one or more weeks or months, for example, and any necessary modifications made accordingly.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for implanting stimulation system 10 into a person for electrical stimulation of the a predetermined site to treat one or more neurological disorders or conditions.

IV. Infusion Pumps

In further embodiments, it may be desirable to use a drug delivery system independent of or in combination with the electrical stimulation systems described herein. Drug delivery may be used independent of or in combination with a lead/electrode to provide electrical stimulation and chemical stimulation. When used, the drug delivery catheter is implanted such that the proximal end of the catheter is coupled to a pump and a discharge portion for infusing a dosage of a pharmaceutical or drug. Implantation of the catheter can be achieved by using techniques well known and used in the art. Thus, without being bound to a specific procedure, implantation of the catheter can be achieved using similar techniques as discussed above for implantation of electrical stimulation portions (e.g., electrical leads and/or electrodes), which is incorporated herein. The distal portion of the catheter can have multiple orifices to maximize delivery of the pharmaceutical while minimizing mechanical occlusion. The proximal portion of the catheter can be connected directly to a pump or via a metal, plastic, or other hollow connector, to an extending catheter.

Any type of infusion pump can be used in the present invention. For example, “active pumping” devices or so-called peristaltic pumps are described in U.S. Pat. Nos. 4,692,147, 5,840,069, and 6,036,459, which are incorporated herein by reference in their entirety. Peristaltic pumps are used to provide a metered amount of a drug in response to an electronic pulse generated by control circuitry associated within the device. An example of a commercially available peristaltic pump is SynchroMed® implantable pump from Medtronic, Inc., Minneapolis, Minn.

Other pumps that may be used in the present invention include accumulator-type pumps, for example certain external infusion pumps from Minimed, Inc., Northridge, Calif. and Infusaid® implantable pump from Strato/Infusaid, Inc., Norwood, Mass. Passive pumping mechanisms can be used to release an agent in a constant flow or intermittently or in a bolus release. Passive type pumps include, for example, but are not limited to gas-driven pumps described in U.S. Pat. Nos. 3,731,681 and 3,951,147; and drive-spring diaphragm pumps described in U.S. Pat. Nos. 4,772,263; 6,666,845; and 6,620,151 which are incorporated by reference in its entirety. Pumps of this type are commercially available, for example, Model 3000® from Arrow International, Reading, Penn. and IsoMed® from Medtronic, Inc., Minneapolis, Minn.; AccuRx® pump from Advanced Neuromodulation Systems, Inc., Plano, Tex.

In certain embodiments, the catheter can be in the form of a lead catheter combination, similar to the ones described in U.S. Pat. No. 6,176,242 and U.S. Pat. No. 5,423,877, which are incorporated herein by reference in their entirety.

V. Identifying a Subject with a Neurological Disorder

In certain embodiments of the invention, subjects to be treated using the present invention can be selected, identified and/or diagnosed based upon the accumulation of physical, chemical, and historical behavioral data on each patient. One of skill in the art is able to perform the appropriate examinations to accumulate such data. One type of examination can include neurological examinations, which can include mental status evaluations, which can further include a psychiatric assessment. Other types of examinations can include, but are not limited to, motor examination, cranial nerve examination, and neuropsychological tests (i.e., Minnesota Multiphasic Personality Inventory, Beck Depression Inventory, or Hamilton Rating Scale for Depression).

In addition to the above examinations, imaging techniques can be used to determine normal and abnormal brain function that can result in disorders. Functional brain imaging allows for localization of specific normal and abnormal functioning of the nervous system. This includes exemplary electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET), which can be utilized to localize brain function and dysfunction.

VI. Methods to Treat Neurological Disorders

The present method acts to stimulate nerve afferents which in turn stimulate the brain and cause/allow the brain to act in the best interest of the host through use of the brain's natural mechanisms. The prior art fails to recognize that stimulation of spinal nervous tissue associated with a C1, C2, or C3 cervical vertebral segment can provide the therapeutic treatments according to the instant invention.

It may come as a surprise to one skilled in the art to learn that stimulation of at least one of a patient's nerves located in or associated with the spinal nervous tissue associated with a C1, C2, or C3 cervical vertebral segment may be used to treat the maladies disclosed herein. While the normal functions of the nerves associated with the spinal nervous tissue associated with a C1, C2, or C3 cervical vertebral segment would not suggest to one skilled in the art that they could be used to treat, for example, depression, anxiety, cognitive disorders, compulsive disorders, or other neurological disorders disclosed herein, for example, the nerves associated with the spinal nervous tissue associated with a C1, C2, or C3 cervical vertebral segment have qualities that make them suited for the method of the invention.

Accordingly, the present invention relates to modulation of neuronal activity to affect neurological, neuropsychological or neuropsychiatric activity. The present invention finds particular application in the modulation of neuronal function or processing to effect a functional outcome. The modulation of neuronal function is particularly useful with regard to the prevention, treatment, or amelioration of neurological, psychiatric, psychological, conscious state, behavioral, mood, and thought activity (unless otherwise indicated these will be collectively referred to herein as “neurological activity” which includes “psychological activity” or “psychiatric activity”). When referring to a pathological or undesirable condition associated with the activity, reference may be made to a neurological disorder that includes “psychiatric disorder” or “psychological disorder” instead of neurological activity or psychiatric or psychological activity. Although the activity to be modulated usually manifests itself in the form of a disorder, such as attention or cognitive disorders (e.g., Autistic Spectrum Disorders); mood disorder (e.g., major depressive disorder, bipolar disorder, and dysthymic disorder); an anxiety disorder (e.g., panic disorder, posttraumatic stress disorder, obsessive-compulsive disorder and phobic disorder); and/or neurodegenerative diseases (e.g., multiple sclerosis, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), one skilled in the art appreciates that the invention may also find application in conjunction with enhancing or diminishing any neurological or psychiatric function, not just an abnormality or disorder. Neurological activity that may be modulated can include, but not be limited to, normal functions such as alertness, conscious state, drive, fear, anger, anxiety, repetitive behavior, impulses, urges, obsessions, euphoria, sadness, memory, and the fight or flight response.

In certain embodiments, neurological disorders or conditions that can be treated using the present invention include, for example, but are not limited to, cardiovascular diseases, e.g., atherosclerosis, coronary artery disease, hypertension, hyperlipidemia, cardiomyopathy, volume retention; neurodegenerative diseases, e.g., Alzheimer's disease, Pick's disease, dementia, delirium, Parkinson's disease, amyotrophic lateral sclerosis; neuroinflammatory diseases, e.g., viral meningitis, viral encephalitis, fungal meningitis, fungal encephalitis, multiple sclerosis, charcot joint; myasthenia gravis; orthopedic diseases, e.g., osteoarthritis, inflammatory arthritis, reflex sympathetic dystrophy, Paget's disease, osteoporosis; lymphoproliferative diseases, e.g., lymphoma, lymphoproliferative disease, Hodgkin's disease; autoimmune diseases, e.g., Graves disease, hashimoto's, takayasu's disease, kawasaki's diseases, arthritis, scleroderma, CREST syndrome, allergies, dermatitis, Henoch-schlonlein purpura, goodpasture syndrome, autoimmune thyroiditis, myasthenia gravis, Reiter's disease, lupus, rheumatoid arthritis; inflammatory and infectious diseases, e.g., sepsis, viral and fungal infections, wound healing, tuberculosis, infection, human immunodeficiency virus; pulmonary diseases, e.g., tachypnea, fibrotic diseases such as cystic fibrosis, interstitial lung disease, desquamative interstitial pneumonitis, non-specific interstitial pneumonitis, lymphocytic interstitial pneumonitis, usual interstitial pneumonitis, idiopathic pulmonary fibrosis; transplant related side effects such as rejection, transplant-related tachycardia, renal failure, typhlitis; transplant related bowel dysmotility, transplant-related hyperreninemia; sleep disorders, e.g., insomnia, obstructive sleep apnea, central sleep apnea; gastrointestinal disorders, e.g., hepatitis, xerostomia, bowel dysmotility, peptic ulcer disease, constipation, post-operative bowel dysmotility; inflammatory bowel disease; endocrine disorders, e.g., hypothyroidism, hyperglycemia, diabetes, obesity, syndrome X; cardiac rhythm disorders, e.g., sick sinus syndrome, bradycardia, tachycardia, QT interval prolongation arrhythmias, atrial arrhythmias, ventricular arrhythmias; genitourinary disorders, e.g., bladder dysfunction, renal failure, hyperreninemia, hepatorenal syndrome, renal tubular acidosis, erectile dysfunction; cancer; fibrosis; skin disorders, e.g., wrinkles, cutaneous vasculitis, psoriasis; aging associated diseases and conditions, e.g., shy dragers, multi-system atrophy, osteoporosis, age related inflammation conditions, degenerative disorders; autonomic dysregulation diseases; e.g., headaches, concussions, post-concussive syndrome, coronary syndromes, coronary vasospasm; neurocardiogenic syncope; neurologic diseases such as epilepsy, seizures, stress, bipolar disorder, migraines and chronic headaches; conditions related to pregnancy such as amniotic fluid embolism, pregnancy-related arrhythmias, fetal stress, fetal hypoxia, eclampsia, preeclampsia; conditions that cause hypoxia, hypercarbia, hypercapnia, acidosis, acidemia, such as chronic obstructive lung disease, emphysema, cardiogenic pulmonary edema, non-cardiogenic pulmonary edema, neurogenic edema, pleural effusion, adult respiratory distress syndrome, pulmonary-renal syndromes, interstitial lung diseases, pulmonary fibrosis, and any other chronic lung disease; sudden death syndromes, e.g., sudden infant death syndrome, sudden adult death syndrome; vascular disorders, e.g., acute pulmonary embolism, chronic pulmonary embolism, deep venous thrombosis, venous thrombosis, arterial thrombosis, coagulopathy, aortic dissection, aortic aneurysm, arterial aneurysm, myocardial infarction, coronary vasospasm, cerebral vasospasm, mesenteric ischemia, arterial vasospasm, malignant hypertension; primary and secondary pulmonary hypertension, reperfusion syndrome, ischemia, cerebral vascular accident, cerebral vascular accident and transient ischemic attacks; pediatric diseases such as respiratory distress syndrome; bronchopulmonary dysplasia; Hirschprung disease; congenital megacolon, aganglionosis; ocular diseases such as glaucoma; and the like. Other disease and/or conditions that may be treated using the present invention are further described in U.S. Patent Publication No. 20040249416, which is hereby incorporated by reference in its entirety.

The present invention finds particular utility in its application to human psychological or psychiatric activity/disorder. However, it is also to be appreciated that the present invention is applicable to other animals that exhibit behavior that is modulated by the brain. This may include, for example, rodents, primates, canines, felines, elephants, dolphins, etc. Utilizing the various embodiments of the present invention, one skilled in the art may be able to modulate the functional outcome of the brain to achieve a desirable result.

Treatment regimens may vary as well, and often depend on the health and age of the patient. Obviously, certain types of disease will require more aggressive treatment, while at the same time, certain patients cannot tolerate more taxing regimens. The skilled artisan will be best suited and is suitably skilled to make such decisions based on the known subject's history.

The therapeutic system of the present invention is surgically implanted in the subject's body as described herein. One of skill in the art is cognizant that a variety of electrodes or electrical stimulation leads may be utilized in the present invention. It is desirable to use an electrode or lead that contacts or conforms to the target site for optimal delivery of electrical stimulation. One such example, is a single multi contact electrode with eight contacts separated by 2½ mm each contract would have a span of approximately 2 mm. Another example is an electrode with two 1 cm contacts with a 2 mm intervening gap. Yet further, another example of an electrode that can be used in the present invention is a 2 or 3 branched electrode to cover the target site. Each one of these three pronged electrodes have four contacts 1-2 mm contacts with a center to center separation of 2 of 2.5 mm and a span of 1.5 mm

According to one embodiment of the present invention, the target site is stimulated using stimulation parameters, such as pulse width of about 1 to about 500 microseconds, more preferable about 1 to about 90 microseconds; frequency of about 1 to about 300 Hz, more preferably, about 100 to about 185 Hz; and voltage of about 0.5 to about 10 volts, more preferably about 1 to about 10 volts. It is known in the art that the range for the stimulation parameters may be greater or smaller depending on the particular patient needs and can be determined by the skilled artisan. Other parameters that can be considered may include the type of stimulation for example, but not limited to acute stimulation, subacute stimulation, and/or chronic stimulation.

Using the stimulation system of the present invention, the predetermined site or target area is stimulated in an effective amount or effective treatment regimen to decrease, reduce, modulate or abrogate the neurological disorder. Thus, a subject is administered a therapeutically effective stimulation so that the subject has an improvement in the parameters relating to the neurological disorder or condition including subjective measures such as, for example, neurological examinations and neuropsychological tests (e.g., Minnesota Multiphasic Personality Inventory, Beck Depression Inventory, Mini-Mental Status Examination (MMSE), Hamilton Rating Scale for Depression, Wisconsin Card Sorting Test (WCST), Tower of London, Stroop task, MADRAS, CGI, N-BAC, or Yale-Brown Obsessive Compulsive score (Y-BOCS)), motor examination, and cranial nerve examination, and objective measures including use of additional psychiatric medications, such as anti-depressants, or other alterations in cerebral blood flow or metabolism and/or neurochemistry.

Patient outcomes may also be tested by health-related quality of life (HRQL) measures: patient outcome measures that extend beyond traditional measures of mortality and morbidity, to include such dimensions as physiology, function, social activity, cognition, emotion, sleep and rest, energy and vitality, health perception, and general life satisfaction, for example. (Some of these are also known as health status, functional status, or quality of life measures.)

Functional imaging may also be used to measure the effectiveness of the treatment. This includes electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET) that can be utilized to localize brain function and dysfunction. Also, electrophysiological examinations, such as electromyography (EMG) and nerve conduction studies (NCS), can also be utilized to assess the effectiveness of the treatment.

Clinical observations indicate that the efficacy of treatment may be correlated to the amplitude or intensity; that is, the higher the amplitude or intensity, the more pronounced the therapeutic effect. Also, unlike certain other types of stimulation such as electrical stimulation of the spinal cord to treat pain, with electrical stimulation of the neuronal tissue in the spinal nervous tissue associated with a C1, C2, or C3 cervical vertebral segment it is generally not necessary for the patient to feel the electrical stimulation to experience the therapeutic effect. When the amplitude or intensity of the electrical stimulation is increased such that the patient can again feel the electrical stimulation, the patient may experience a further amplification of the beneficial effects. After a time (e.g., approximately thirty minutes) being stimulated at the increased amplitude or intensity, the ability of the patient to feel the electrical stimulation again fades. In certain embodiments, this phenomenon may allow the amplitude or intensity to be increased more or less indefinitely to achieve increased beneficial effects.

For purposes of this invention, beneficial or desired clinical results include, but are not limited to, alleviation of symptoms, improvement of symptoms, diminishment of extent of disease, stabilized (i.e., not worsening) state of disease, delay or slowing of disease progression, amelioration or palliation of the disease state, and remission (whether partial or total), whether objective or subjective.

In certain embodiments, in connection with improvement in one or more of the above or other neurological disorders, the electrical stimulation may have a “brightening” effect on the person such that the person looks better, feels better, moves better, thinks better, and/or otherwise experiences an overall improvement in quality of life.

In certain embodiments, electrical stimulation of the spinal nervous tissue associated with a C1, C2, or C3 cervical vertebral segment may be provided to effectively treat pain. For example, in certain embodiments, electrical stimulation of the spinal nervous tissue associated with a C1, C2, or C3 cervical vertebral segment may be provided to effectively treat fibromyalgia or other diffuse pain in any one or more regions of the body.

In certain embodiments, electrical stimulation of the spinal nervous tissue associated with a C1, C2, or C3 cervical vertebral segment may effectively treat one or more neurological disorders associated with traumatic brain injury (TBI). Physiological conditions associated with TBI that may be treated effectively through electrical stimulation of the spinal nervous tissue associated with a C1, C2, or C3 cervical vertebral segment include, for example, intractable localized, diffuse, or other pain in the head, neck, shoulders, upper extremities, and/or low back, fibromyalgia or other diffuse pain in one or more regions of the body, and/or other pain symptoms. Instead of or in addition to such physiological conditions, psychological, and other conditions associated with TBI that may be treated effectively through electrical stimulation of the spinal nervous tissue associated with a C1, C2, or C3 cervical vertebral segment include, for example, intractable nausea (e.g., from gastroparesis), sleep disorders, chronic fatigue, behavioral modifications (e.g., lassitude, reduced motivation, depression, emotional distress, irritability, aggression, anxiety, erratic mood swings, personality changes, and loss of enjoyment), sexual dysfunction, and other conditions. Instead of or in addition to physiological, psychological, and other conditions such as those described above, conditions associated with TBI that may be treated effectively through electrical stimulation of the spinal nervous tissue associated with a C1, C2, or C3 cervical vertebral segment include, for example decreased cognitive functioning in the form of, for example, impaired memory (e.g., short-term memory, visual memory, and auditory memory), reduced attention and concentration, and/or reduced information processing capacity (e.g., learning capacity, ability to process complex information, ability to operate simultaneously on different information, ability to rapidly shift attention, ability to plan and sequence, visuomotor capability, auditory language comprehension, and/or verbal fluency).

VII. Hypertension

The present invention provides novel methods of treating one or more neurological disorders and conditions by stimulating neuronal tissue associated with a cervical vertebral segment. For example, a patient with hypertension is treated with spinal cord stimulation of spinal cord or neuronal tissue associated with a C1, or C2 vertebral segment by surgical insertion of a stimulation system as described inserted in accordance with the present invention and as is known to those skilled in the art. The stimulation system is implanted and delivers electrical stimulation to spinal cord or neuronal tissue associated with a C1, C2, or C3 vertebral segment. The stimulation relieves hypertension associated with the patient's condition. The stimulation also increases the patient's blood flow to the brain. In certain embodiments of the invention, the stimulation of cervical spinal cord nervous tissue associated with C1, C2, or C3 causes vasodilation of blood vessels.

Hypertension, or elevated blood pressure, is a relatively common affliction. A 1993 Canadian study of 1,374 individuals ranging from 30 to 69 years of age found that 32% of the male adults and 19% of the female adults in the study exhibited high blood pressure. Most patients with hypertension exhibit the hemodynamic abnormality of increased vascular resistance. Treatment is essential to limit secondary organ damage to the heart, kidneys and eyes, and other effects which tend to contribute to early death of the hypertensive person.

The general term, “blood pressure” applies to arterial blood pressure in the circulation system. It fluctuates with each heart beat between a systolic maximum level during contraction and a minimum pressure during its diastolic phase. The geometric mean value is known as the pulse pressure of a human or animal.

Blood vessels are muscles which are constricted or dilated to provide correct blood circulation performance. As part of this performance, control of the heart is also modulated as to beat rate and myocardial contractile tone. Information sent to the brain regarding performance status is provided by afferent sensors that span the body. Such afferent sensors can be chemical, mechanical, thermal and pressure receptors that provide minute low voltage informational signals to the brain. Such signals can be from outside the body as provided by auditory or visual afferent sensors or internal sensors located within the cardiovascular system and elsewhere. In addition to the electrical signals from the brain, neurotransmitter hormones produced at nerve synapses or the endocrine system modulate blood pressure.

As the heart contracts and pumps blood (systole), the arteries stretch and store potential energy. When the heart relaxes (diastole) the arteries rebound and keep the blood flowing. This is called the “windkessel” effect and assures continuing circulation to supply of oxygen and nutrients to all parts of the body between heartbeats (contractions).

Regulation of blood flow to the various organs is mainly achieved by alterations in the diameter of the blood vessel lumen (inside bore). The lumen can be incrementally constricted or dilated as required. This lumenal control is accomplished by chemical effects and neural instructions coming from the brain. Blood vessels consist of smooth muscle and contain electrically active cells that continually vary between constriction and relaxation. Nervous control of the blood vessels is mediated with only a few exceptions by the sympathetic nerves of the autonomic nervous system. The autonomic nerves are regulated without conscious participation of the individual.

In the arterial high pressure control side there are stretch and pressure receptor afferent nerves from the aorta and carotid arteries to provide key information. In the low pressure venous system stretch and other receptors located in the vena cava, atrial heart chambers and in the left ventricle provide blood pressure pulse rate and filling pressure data to the brains medullopontine. Afferent sensory data which compute into efferent nerve signals back to the cardiovascular system is processed in various nucleus tracts of the medulla oblongata and its olive. Alterations in newly arriving afferent data is compared to existing efferent control output before modulative corrective responses are elicited and sent off to the heart and blood vessels.

The nucleus of the solitary tract (NTS), a termination site for primary afferent fibers from baroreceptors and other peripheral cardiovascular receptors, and the paratrigeminal nucleus (Pa5) contain blood pressure-sensitive neurons, some of which have rhythmic activity locked to the cardiac cycle, making them key components of the central pathway for cardiovascular regulation. NTS and Pa5 baroreceptor-activated neurons possess phasic discharge patterns locked to the cardiac cycle (Junior, Caous et al., 2004). The human insular cortex is involved in cardiac regulation. The left insula is predominantly responsible for parasympathetic cardiovascular effects. On stimulation of the left insular cortex, parasympathetic tone increases resulting in bradycardia and depressor responses more frequently than tachycardia and pressor effects (p<0.005) (Oppenheimer, Gelb et al., 1992). The converse applies for the right insular cortex: stimulation of the human right insula increases sympathetic cardiovascular tone (Oppenheimer 1993). Acute left insular stroke increases basal cardiac sympathetic tone and is associated with a decrease in randomness of heart rate variability (Oppenheimer, Kedem et al., 1996). Increased sympathoadrenal tone, resulting from damage to cortical areas involved in cardiac and autonomic control can induce cardiac damage by nonischemic mechanisms (Oppenheimer and Hachinski 1992).

The autonomic nervous system plays an important role in the genesis of various cardiac rhythm disorders. In patients with paroxysmal atrial fibrillation, it is important to distinguish vagally mediated from adrenergically mediated atrial fibrillation. The former is considered to represent a form of lone atrial fibrillation affecting particularly males aged 40 to 50 years. The arrhythmic episodes manifest themselves most often during the night lasting from minutes to hours, whereas in adrenergic mediated atrial fibrillation, atrial fibrillation is often provoked by emotional or physical stress. (Hohnloser, van de Loo et al., 1994)

Thus, hypertension (e.g., neurogenic hypertension) can be treated with the stimulation of spinal nervous tissue of the present invention.

VIII. Combination Treatment

In some embodiment of the invention, an under recurring the electrical stimulation of the invention is also administered an additional treatment. In specific embodiments, in order to increase the effectiveness of the electrical stimulation method of the present invention, it may be desirable to combine electrical stimulation with chemical stimulation to treat the neurological condition.

In one preferred alternative, an implantable pulse generation source and electrical stimulating portion and an implantable pump and catheter(s) are used to deliver electrical stimulation and/or one or more stimulating drugs to the above mentioned areas as a treatment for mood and/or anxiety disorders.

Herein, stimulating drugs comprise medications, anesthetic agents, synthetic or natural peptides or hormones, neurotransmitters, cytokines and other intracellular and intercellular chemical signals and messengers, and the like. In addition, certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug. In addition, stimulation of an area herein includes stimulation of cell bodies and axons in the area.

Similarly, excitatory neurotransmitter agonists (e.g., norepinephrine, epinephrine, glutamate, acetylcholine, serotonin, dopamine), agonists thereof, and agents that act to increase levels of an excitatory neurotransmitter(s) (e.g., edrophonium; Mestinon; trazodone; SSRIs (e.g., flouxetine, paroxetine, sertraline, citalopram and fluvoxamine); tricyclic antidepressants (e.g., imipramine, amitriptyline, doxepin, desipramine, trimipramine and nortriptyline), monoamine oxidase inhibitors (e.g., phenelzine, tranylcypromine, isocarboxasid)), generally have an excitatory effect on neural tissue, while inhibitory neurotransmitters (e.g., dopamine, glycine, and gamma-aminobutyric acid (GABA)), agonists thereof, and agents that act to increase levels of an inhibitory neurotransmitter(s) generally have an inhibitory effect. (Dopamine acts as an excitatory neurotransmitter in some locations and circumstances, and as an inhibitory neurotransmitter in other locations and circumstances.) However, antagonists of inhibitory neurotransmitters (e.g., bicuculline) and agents that act to decrease levels of an inhibitory neurotransmitter(s) have been demonstrated to excite neural tissue, leading to increased neural activity. Similarly, excitatory neurotransmitter antagonists (e.g., prazosin, and metoprolol) and agents that decrease levels of excitatory neurotransmitters may inhibit neural activity. Yet further, lithium salts and anesthetics (e.g., lidocane) may also be used in combination with electrical stimulation.

In addition to electrical stimulation and/or chemical stimulation, other forms of stimulation can be used, for example magnetic, or thermal or combinations thereof. Magnetic stimulation can be provided by internally implanted probes or by externally applied directed magnetic fields, for example, U.S. Pat. Nos. 6,592,509; 6,132,361; 5,752,911; and 6,425,852, each of which is incorporated herein in its entirety. Thermal stimulation can be provided by using implanted probes that are regulated for heat and/or cold temperatures which can stimulate or inhibit neuronal activity, for example, U.S. Pat. No. 6,567,696, which is incorporated herein by reference in its entirety.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for implanting an example stimulation system 10 into a person for electrical stimulation of the spinal cord.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

We claim:

1. A method of treating a patient with a neurological disorder or condition comprising the steps of:
surgically implanting in the patient a stimulation system in communication with spinal nervous tissue at one or more areas associated with the first, second, or third cervical vertebral segment;
stimulating the spinal nervous tissue by operating the system; and
treating the neurological disorder or condition with the stimulation.

2. The method of claim 1, wherein the stimulating comprises electrical stimulation.

3. The method of claim 1, wherein the stimulating comprises chemical stimulation.

4. The method of claim 1, wherein the neurological disorder or condition is further defined as an affective disorder.

5. The method of claim 1, wherein the neurological disorder or condition is further defined as a mood disorder.

6. The method of claim 1, wherein the neurological disorder or condition is further defined as a sleep disorder.

7. The method of claim 1, wherein the neurological disorder or condition is further defined as a motor disorder.

8. The method of claim 7, wherein the motor disorder is selected from the group consisting of comprise tremor, dystonia, and seizure.

9. The method of claim 1, wherein the neurological disorder or condition is further defined as epilepsy.

10. The method of claim 1, wherein the neurological disorder or condition is further defined as Parkinson's Disease.

11. The method of claim 1, wherein the neurological disorder is a decreased in cognitive functioning.

12. The method of claim 11, wherein the decreased cognitive functioning comprises one or more impaired memory, reduced attention and concentration, and reduced information processing capacity.

13. The method of claim 1, wherein the neurological disorder is selected from the group consisting of intractable nausea, chronic fatigue, and migraine headaches.

14. The method of claim 1, wherein the spinal nervous tissue is associated with the first cervical vertebral segment.

15. The method of claim 1, wherein the spinal nervous tissue is associated with the second vertebral segment.

16. The method of claim 1, wherein the spinal nervous tissue is associated with the third vertebral segment.

17. The method of claim 1, wherein the system comprises an electrode.

18. The method of claim 1, wherein the system allows the patient to control the frequency of stimulation.

19. The method of claim 1, wherein the stimulation is noncontinuous.

20. The method of claim 1, further comprising the step of assessing the neurological disease or condition in the patient after the stimulation.

21. A method of increasing blood flow to the brain comprising the steps of:
surgically implanting a stimulation system in communication with spinal nervous tissue at one or more areas associated with the first, second, or third cervical vertebral segment;
operating the system to stimulate the spinal nervous tissue; and
increasing blood flow to the brain.

22. The method of claim 22, further comprising the step of assessing the blood flow to the brain.

23. A method of treating hypertension in a patient comprising the steps of:
surgically implanting in the patient a stimulation system in communication with spinal nervous tissue at one or more areas associated with the first, second, or third cervical vertebral segment;
operating the system to stimulate the spinal nervous tissue; and
treating hypertension in the patient.

24. A method of treating a migraine headache in a patient comprising the steps of:
surgically implanting in the patient a stimulation system in communication with spinal nervous tissue at one or more areas associated with the first, second, or third cervical vertebral segment;
operating the system to stimulate the spinal nervous tissue; and
treating the migraine headache in the patient.

25. A method of treating a neurological disorder and/or condition comprising the steps of:
surgically implanting at least one electrode that is in communication with spinal nervous tissue at one or more areas associated with the first, second, or third cervical vertebral segment;
coupling the electrode to a pulse generating source; and
generating an electrical signal with the pulse generating source wherein said signal electrically stimulates the spinal nervous tissue, thereby treating the neurological disorder and/or condition.

26. The method of claim 25 further comprising the steps of:
surgically implanting a catheter having a proximal end coupled to a pump and a discharge portion for infusing a dosage of a pharmaceutical, wherein after implantation the discharge portion of the catheter is in communication with the spinal nervous tissue; and
operating the pump to discharge the pharmaceutical through the discharge portion of the catheter into the spinal nervous tissue thereby treating the neurological disorder and/or condition.

27. The method of claim 25, wherein the stimulation results in modulation of blood flow to the brain.

28. The method of claim 25, wherein the neurological disorder and/or condition is selected from the group consisting of Developmental Disabilities, Pervasive Developmental Disorders and Autistic Spectrum Disorders, nonverbal learning disabilities, Demyleinating Diseases, delirium and dementia, affective disorder, movement disorders, ataxic disorders, substance abuse related disorders, sexual dysfunctions, eating disorders, anxiety and obsessive compulsive disorder syndromes, impulse control disorders, personality disorders, and Chiari I malformation.

29. A stimulation system for treating a neurological disorder and/or condition comprising:
at least two electrodes operatively connected in a single unit to a signal generator, the electrodes in communication with spinal nervous tissue at one or more areas associated with the first, second, or third cervical vertebral segment, thereby stimulating the spinal nervous tissue, and treating the neurological disorder and/or condition.

30. A stimulation system for treating a neurological disorder and/or condition comprising:
at least one electrode in communication with spinal nervous tissue at one or more areas associated with the first, second, or third cervical vertebral segment; and
a pulse generation source operatively connected to at least one, thereby stimulating the spinal nervous tissue, and treating the neurological disorder and/or condition.

31. The system of claim 30, wherein the pulse generation source an electrical signal.

32. A system for treating subjects with neurological disorders and/or conditions comprising:
at least one electrode that is implanted into the subject's spinal nervous tissue, the least one electrode is in communication with spinal nervous tissue at one or more areas associated with the first, second, or third cervical vertebral segment;
a pulse generation source that generates signals for transmission to the at least one electrode resulting in delivery of electrical signals to the spinal nervous tissue thereby treating the neurological disorder and/or conditions.

33. The system of claim 32, wherein the pulse generation source is implanted in the subject's body.
